(19) United States
(12) Patent Application Publication
(10) Pub. No.: US 2013/0316949 A1
(43) Pub. Date: Nov. 28, 2013

(54) Compositions and Methods for Minimally-Invasive Systemic Delivery of Proteins Including TGF-Beta Superfamily Members

(71) Applicant: STRYKER CORPORATION, (US)

(72) Inventors: Mary Elizabeth Pecquet Goad, Andover, MA (US); Denis Schrier, Hopkinton, MA (US); Allen Richard Pierce, Acton, MA (US)

(73) Assignee: STRYKER CORPORATION, Kalamazoo, MI (US)

(21) Appl. No.: 13/707,717

(22) Filed: Dec. 7, 2012

Related U.S. Application Data
(63) Continuation of application No. 13/444,257, filed on Apr. 11, 2012, now abandoned.
Application No. 13/444,257 is a continuation of application No. 12/705,367, filed on Feb. 12, 2010, now abandoned.
(60) Provisional application No. 61/151,902, filed on Feb. 12, 2009.

(51) Int. Cl. A61K 38/18

(52) U.S. Cl. CPC A61K 38/1875
USPC 514/8.8

(57) ABSTRACT

The present invention is directed to methods and compositions for systemic delivery of minimally-soluble bioactive agents such as, but not limited to, proteins of the TGF-β superfamily. According to the invention, an exemplary bioactive agent is BMP-7 The invention further provides for minimally-invasive systemic treatment of skeletal disorders such as osteoporosis as well as minimally-invasive systemic treatment of injured or diseased non-mineralized tissues and organs such kidneys. Practice of the invention eliminates adverse side effects at the site of intravascular delivery of the bioactive agent.

### CROSS-REFERENCE TO RELATED APPLICATIONS

This application claims priority to and the benefit of U.S. Provisional Patent Application No. 61/151,902, filed Feb. 12, 2009, the contents of which are incorporated by reference herein.

### BACKGROUND

Bone morphogenetic proteins (BMPs) belong to the superfamily of transforming growth factor β (TGF-β), and control a diverse set of cellular and developmental processes, such as pattern formation and tissue specification as well as promoting wound healing and repair processes in adult tissues. BMPs were initially isolated by their ability to induce bone and cartilage formation; however, their utility for other tissue and organ repair is now widely appreciated.

To date, a reliable means for non-local delivery of a clinically effective dose of a BMP—especially over a prolonged period of time, without repeated administration of the BMP—has eluded the skilled practitioner. In fact, effective delivery of most proteinaceous biologic agents generally remains an unanswered challenge. Despite progress in protein technologies and pharmaceutical chemistries, at least two problems continue to plague clinicians needing to provide key physiological factors to patients.

First, the preferred mode of administration of most therapeutic agents is oral or by injection. However, oral administration is often inappropriate for macromolecular drugs such as proteins, as many of them are unstable in the gastrointestinal tract which can compromise the efficacy of a particular dosage regimen. Moreover, when a particular therapeutic protein or proteinaceous agent is administered by routine injection methods, frequent and multiple injections are required because these agents can have negligible bio-availability. Thus, the most popular and routine means of administering medications can pose a substantial physical burden on the patient and create significant administrative costs related to patient management.

Thus, there is a need for alternative modes of providing biologically active agents, especially macromolecules such as BMPs and other proteinaceous macromolecular biologics or drugs.

### SUMMARY OF THE INVENTION

The present invention is based on the discovery that an exemplary bone morphogenetic protein (BMP), BMP-7, can be provided non-surgically and non-locally to mammals without adverse effects by providing a solution of the protein, for example, via a vascular access structure such as but not limited to a central venous catheter. In fact, the invention exploits the discovery that certain specific physiological criteria are determinative in successful administration and delivery. As set forth herein, an exemplary protein, BMP-7, can be provided safely to a human subject suffering from a condition treatable with a BMP by providing a solution of the BMP via a vascular access structure such as a central venous line, a central venous catheter or an arteriovenous fistula to name but a few. For example, a central venous line can include a central venous access catheter inserted into the neck, chest or groin to access, for example, the external or internal jugular vein (neck), the subclavian vein (chest), femoral vein (groin), or superior vena cava. Preferably, a vascular line is placed such that it is easily accessible and the catheter access has sufficiently healed into place; for example, a vascular access port at the injection end of the catheter in which the therapeutic agent is introduced. The vascular line can be placed with or without surgery and then be allowed to heal to minimize or avoid any leakage at the puncture site into the blood vessel. As is explained elsewhere herein, aspects of the invention further include a protein formulation suitable for minimally-invasive systemic delivery, for example, centrally, including formulation parameters such as pH, excipients and/or concentration to name but a few, as well as the rate of administration of such formulations and effective dosages of the same accomplished via manipulation of formulation parameters and/or rates of administration.

While current clinical applications of proteins such as BMPs, as well as other members of the TGF-β superfamily of tissue morphogens, are limited to local, surgically-invasive implantation for inducing local bone growth and repair, preclinical research confirms a number of systemic disease states for which BMP therapy can be beneficial. These include but are not limited to applications in chronic and acute kidney disease, atherosclerosis, pulmonary fibrosis, obesity, diabetes, cancer, ocular scarring, liver fibrosis, inflammatory disorders and nervous system disorders. In accordance with the treatment of such diseases using the present invention, non-local administration of BMP-7 is now appreciated to be the optimal approach. Moreover, the present invention further confirms that conventional methods of systemic administration, such as direct peripheral injection (e.g., via intravascular, subcutaneous or intraperitoneal administration; further including intravascular administration using a syringe equipped with a traditional syringe needle) can have undesirable effects, including the formation of ectopic bone and/or fibrous tissue at the injection site and/or inducement of localized tissue trauma such as for example peripheral edema. As is explained elsewhere herein, the present invention relates to heretofore-undescribed materials and methods for minimally-invasive systemic delivery of a biologic agent, especially a proteinaceous macromolecule such as but not limited to a BMP. It is further understood that minimally-invasive systemic delivery as contemplated herein does not include oral, parenteral or topical delivery.

In a first aspect, the present invention is directed to a composition comprising a biologic agent and a vascular access structure. In certain preferred embodiments, a biologic agent is a minimally soluble protein. In one embodiment, the proteinaceous biologic agent is a protein that is substantially insoluble at physiological pH. In one embodiment, the proteinaceous biologic agent is a member of the TGF-β superfamily of proteins. Another embodiment of the present invention provides for a proteinaceous biologic agent that is a member of the BMP subfamily of the TGF-β superfamily of proteins. In one embodiment of the present invention, the proteinaceous biologic agent is BMP-2, BMP-4, BMP-5, BMP-6, BMP-7, GDF-5, GDF-6, or GDF-7. In another aspect of the present invention, the proteinaceous biologic agent is BMP-7. The present invention also provides for a proteinaceous biologic agent that is sequence variant of any one of BMP-2, BMP-4, BMP-5, BMP-6, BMP-7, GDF-5, GDF-6, or GDF-7. In another aspect of the present invention, the proteinaceous biologic agent is a protein having at least about 50% amino acid sequence identity with a member of the BMP subfamily within the conserved C-terminal cysteine-rich domain.

In accordance with the present invention, a vascular access structure is a device or apparatus which provides access to a subject's vasculature. As contemplated by the present invention, a vascular access structure is implantable on the exterior or the interior of a subject. According to the invention, the vasculature may be accessed in one embodiment peripherally, or in another embodiment, centrally. According to one embodiment of the invention, the delivery site of a biologic agent into the subject's blood stream occurs centrally, for example, via an external or internal jugular vein, subclavian vein, femoral vein, or superior vena cava. In certain embodiments, such a vascular access structure can operate to deliver a biologic agent into the subject's blood stream at a site remote from the actual puncture site. A central delivery site is preferable. It is also preferable that the actual site of puncture into the blood vessel itself is allowed to heal prior to the introduction of biologic agent, such as a BMP, as is typical with placement of a catheter with a vascular access port. This minimizes or avoids leakage of agent at the direct site of puncture of the vasculature.

In a currently preferred embodiment, a composition of the present invention suitable for ameliorating an injury or disease comprises a biologic agent selected from the group consisting of: a member of the TGF-β superfamily of proteins, a member of the BMP subfamily of the TGF-β superfamily of proteins, and a protein having at least about 50% amino acid sequence identity with a member of the BMP subfamily within the conserved C-terminal cysteine-rich domain; and, a vascular access structure selected from the group consisting of: a central venous catheter, a central venous line, a subcutaneous port, and structures having functionally or structurally similar configurations thereto, wherein said biologic agent is in an amount effective to ameliorate an injury or disease.

In another aspect, the present invention also provides for a formulation comprising a biologic agent in amount effective to ameliorate tissue injury or disease which is suitable for inclusion with the compositions described above. In one embodiment, the injury to be ameliorated is a mineralized or non-mineralized skeletal tissue injury. In another embodiment, the injury or disease to be ameliorated is metabolic bone disease, osteoarthritis, osteochondral disease, rheumatoid arthritis, osteoporosis, Paget's disease, periodontitis, dentinogenesis, chondral disease, trauma-induced and inflammation-induced cartilage degeneration, age-related cartilage degeneration, articular cartilage injuries and diseases, full thickness cartilage diseases, superficial cartilage defects, sequelae of systemic lupus erythematosis, sequelae of scleroderma, periodontal tissue regeneration, herniation and rupture of intervertebral discs, degenerative diseases of the intervertebral disc, osteocondrosis, or injuries and diseases of ligament, tendon, synovial capsule, synovial membrane and meniscal tissues. In another embodiment, the injury or disease to be ameliorated is liver disease, liver resection, hepatectomy, renal disease, chronic renal failure, central nervous system ischemia or trauma, neuropathy, motor neuron injury, dendritic cell deficiencies and abnormalities, Parkinson's disease, ophthalmic disease, ocular scarring, retinal scarring, or ulcerative diseases of the gastrointestinal tract. In yet another embodiment, the composition comprises biologic agent is in an amount effective to suppress tumor cell proliferation or promote tumor regression.

In yet another aspect, the present invention contemplates methods of systemic treatment using proteins such as but not limited to those of the TGF-β superfamily which are miniminally invasive. As used herein, “systemic” means non-local. The skilled practitioner will understand that non-local can include a method whereby a protein or other bioactive agent is introduced to a subject at a single local site, such as but not limited to a peripheral percutaneous site, so as to effectuate treatment of the subject's whole body rather than just a single local site. In a further embodiment, “systemic” can also mean that therapeutic blood levels of an administered therapeutic agent are present in the blood at a point in time. “Systemic” administration can also effectuate treatment of a site in a patient's body remote from the site of administration by providing therapeutic blood levels of an administered therapeutic agent. As used herein, “minimally-invasive” means non-invasive or non-open-field surgical methods. The skilled practitioner will understand that such methods can include procedures involving an incision(s) or implantation of a medical device(s).

In certain currently preferred embodiments, a method of treatment of an injured or diseased tissue comprises the step of providing to an administration site a composition comprising a biologic agent and a vascular access structure, whereupon the biologic agent is delivered in an amount effective to treat the injured or diseased tissue. Preferably, the physical administration site is remote from the actual site of delivery of the biologic agent. In certain embodiments, the delivery site is a non-peripheral site. For example, the delivery site is a central site. In certain embodiments, the administration site is a peripheral site.

In currently preferred embodiments, upon its delivery, the biologic agent disperses at a rate sufficient to provide a biologically effective dose at a site remote from the site of delivery. For example, in one embodiment, the site of administration is peripheral, while the site of delivery is central. In a particularly preferred embodiment, the biologic agent disperses at a rate of at least 1 ml/min. In another particularly preferred embodiment, the delivery site is substantially edema-free and/or substantially unperturbed and uncompromised. In yet another, the non-vascular tissue at, near or adjacent the delivery site is substantially free of biologic agent following delivery.

In another currently preferred embodiment of the invention, a method of treatment of an injured or diseased tissue comprises administering to an administration site a composition comprising a biologic agent and delivering to an intravascular delivery site the composition such that intima tissue integrity at the delivery site is substantially uncompromised. This would be the case if the initial puncture site of the blood vessel were allowed to heal prior to the administration of a biologic agent, for example a BMP, into the blood vessel lumen. In a preferred embodiment, the biologic agent disperses from the delivery site at a rate and in an amount effective to treat the injured or diseased tissue. In certain embodiments, the administration site and the delivery site are the same. In other embodiments, the delivery site is remote from the administration site. In a particularly preferred embodiment, the delivery site is venular-valve-free. In another, the blood flow rate at the delivery site is sufficient to provide a biologically effective dose at a site remote from the site of delivery. In a particularly preferred embodiment, the biologic agent disperses at a rate of at least 1 ml/min. In yet another, the delivering step is accomplished using an intravascular apparatus having a distal end with a non-damaging configuration. In one such embodiment, the apparatus is equipped with a non-damaging distal end which structurally simulates the non-damaging features of a Foley-type catheter or a functional equivalent thereof.

In keeping with the teachings of the present invention, the currently preferred biologic agent is BMP-7 and injured or diseased non-mineralized tissue is the currently preferred object of treatment. Such injured or diseased tissue can be an organ. In a particularly preferred method of treatment, the biologic agent is bioavailable for at least about 0.5 hours, more preferably at least about 2 hours, at least about 8 hours; for about 1 day, preferably more than 1 day. And, an effective amount is about 10 microgram to about 1000 microgram of biologic agent, more preferably about 50 microgram to about 500 microgram, most preferably about 100 microgram to 300 microgram per kg of patient body weight.

In another embodiment, the invention provides a method of treating a disease in a patient by systemically administering a bone morphogenetic protein to a patient in need thereof. The method includes the step of administering the bone morphogenetic protein to the patient at an administration site via a vascular access structure, wherein the bone morphogenetic protein is delivered to the patient at a centrally located delivery site in the patient. According to another embodiment, the method can further include the step of implanting a vascular access structure with central venous access in the patient. For example, the central venous access can be via the jugular vein, the subclavian vein, the superior vena cava, or the femoral vein according to certain embodiments of the invention. In one embodiment, the vascular access structure is a central venous catheter or central venous port. According to another embodiment of the invention, the administration site is peripheral and the vascular access structure is a PICC line. In an alternative embodiment, the administration site is centrally located. According to one embodiment of the invention, the bone morphogenetic protein is BMP-7. According to one embodiment of the invention, the delivery site is substantially edema free and substantially non-perturbed. In another embodiment, the vascular access structure is substantially healed in place prior to administration of the bone morphogenetic protein.

In another aspect, the present invention also provides for a formulation comprising a biologic agent in amount effective to ameliorate tissue injury or disease which is suitable for use with the methods described above.

In another aspect, the present invention also provides a kit for use in systemically administering a bone morphogenetic protein to a patient in need thereof. In one embodiment, the kit includes a bone morphogenetic protein and a vascular access structure. The kit can further include instructions for systemically administering the bone morphogenetic protein to the patient. For example, the instructions can further indicate that the vascular access structure be implanted in the patient so as to permit central delivery of said bone morphogenetic protein to said patient. According to one embodiment of the invention, the bone morphogenetic protein is BMP-7. The bone morphogenetic protein can be provided in a composition with a suitable pharmaceutical carrier. According to another embodiment of the invention, the vascular access structure is a central venous catheter. In another embodiment, a kit for use in systemically administering a bone morphogenetic protein to a patient in need thereof includes a bone morphogenetic protein and instructions for systemically administering the bone morphogenetic protein to the patient via a centrally located vascular access structure. In a further embodiment, the kit includes a vascular access structure. For example, the vascular access structure provides central administration and delivery of the bone morphogenetic protein.

The foregoing, and other features and advantages of the invention as well as the invention itself, will be more fully understood from the following figures, description, and claims.

### DETAILED DESCRIPTION

The present invention is based on the discovery that an exemplary bone morphogenetic protein (BMP), BMP-7, can be provided non-surgically and non-locally to mammals without adverse effects by providing a solution of the protein, for example, via a vascular access structure such as but not limited to a central venous catheter. In fact, the invention exploits the discovery that certain specific physiological criteria are determinative in successful administration and delivery. As set forth herein, an exemplary protein, BMP-7, can be provided safely to a human subject suffering from a condition treatable with a BMP by providing a solution of the BMP via a vascular access structure such as a central venous line, a central venous catheter or an arteriovenous fistula to name but a few. For example, a central venous line can include a central venous access catheter inserted into the neck, chest or groin to access the jugular vein (neck), the subclavian vein (chest) or femoral vein (groin). Preferably, a vascular line is placed such that it is easily accessible and the catheter access has sufficiently healed into place; for example, a vascular access port at the injection end of the catheter is which the therapeutic agent is introduced. The vascular line can be placed with or without surgery and then be allowed to heal to minimize or avoid any leakage at the puncture site into the blood vessel. As is explained elsewhere herein, aspects of the invention further include a protein formulation, including pH, excipients and/or concentration, as well as the rate of administration and dosage accomplished via modulation of the same.

While current clinical applications of proteins such as BMPs, as well as other members of the TGF-β superfamily of tissue morphogens, are limited to local, surgically-invasive implantation for inducing local bone growth and repair, preclinical research confirms a number of systemic disease states for which BMP therapy can be beneficial. These include but are not limited to applications in metabolic bone diseases including mineralized as well as non-mineralized tissues affected thereby. Additionally, preclinical research confirms a number of systemic disease states for which BMP therapy can be beneficial including tissues and/or organs affected by diseases or disorders such as chronic and acute kidney disease, atherosclerosis, pulmonary fibrosis, obesity, diabetes, cancer, ocular scarring, liver fibrosis, inflammatory disorders and nervous system disorders. In accordance with the treatment of such diseases using the present invention, non-local administration of BMP-7 is now appreciated to be the optimal approach. Moreover, the present invention further confirms that conventional methods of systemic administration, such as direct peripheral injection (e.g., via intravascular, subcutaneous, or intraperitoneal administration) can have undesirable effects, including the formation of ectopic bone and/or fibrous tissue at the site of entry, for example a vascular puncture site, and inducement of localized tissue reactions such as, for example, edema.

### Biologic Agents Including Bone Morphogenetic Proteins

In brief, the present invention contemplates that a suitable biologic agent is preferably a minimally soluble protein. That is, a preferred biologic agent is a protein that is substantially insoluble at physiological pH. For example, an exemplary proteinaceous biologic agent is a member of the TGF-β superfamily of proteins. The present invention further provides for a proteinaceous biologic agent that is a member of the BMP subfamily of the TGF-β superfamily of proteins. In certain embodiments of the present invention, the proteinaceous biologic agent is BMP-2, BMP-4, BMP-5, BMP-6, BMP-7, GDF-5, GDF-6, or GDF-7. In a preferred embodiment of the present invention, the proteinaceous biologic agent is BMP-7. The present invention also provides for a proteinaceous biologic agent that is sequence variant of any one of BMP-2, BMP-4, BMP-5, BMP-6, BMP-7, GDF-5, GDF-6, or GDF-7. In another embodiment of the present invention, the proteinaceous biologic agent is a protein having at least about 50% amino acid sequence identity with a member of the BMP subfamily within the conserved C-terminal cysteine-rich domain.

As stated above, BMPs are a preferred exemplary biologic agent for purposes of the present invention and belong to the TGF-β superfamily. The TGF-β superfamily proteins are cytokines characterized by six-conserved cysteine residues. The human genome contains about 42 open reading frames encoding TGF-β superfamily proteins. The TGF-β superfamily proteins can at least be divided into the BMP subfamily and the TGF-β subfamily biologic agents based on sequence similarity and the specific signaling pathways that they activate. The BMP subfamily includes, but is not limited to, BMP-2, BMP-3 (osteogenin), BMP-3b (GDF-10), BMP-4 (BMP-2b), BMP-5, BMP-6, BMP-7 (osteogenic protein-1 or OP-1), BMP-8 (OP-2), BMP-8B (OP-3), BMP-9 (GDF-2), BMP-10, BMP-11 (GDF-11), BMP-12 (GDF-7), BMP-13 (GDF-6, CDMP-2), BMP-15 (GDF-9), BMP-16, GDF-1, GDF-3, GDF-5 (CDMP-1, MP-52), and GDF-8 (myostatin). For purposes of the present invention, preferred superfamily proteins include BMP-2, -4, -5, -6 and -7 and GDF-5, -6, and -7, as well as MP-52. Particularly preferred proteins include BMP-2, BMP-7 and GDF-5, -6, and -7. A most preferred exemplary BMP is BMP-7. BMPs are also present in other animal species. Furthermore, there is allelic variation in BMP sequences among different members of the human population, and there is species variation among BMPs discovered and characterized to date.

The TGF-β subfamily includes, but is not limited to, TGFs (e.g., TGF-β1, TGF-β2, and TGF-β3), activins (e.g., activin A) and inhibins, macrophage inhibitory cytokine-1 (MIC-1), Mullerian inhibiting substance, anti-Mullerian hormone, and glial cell line derived neurotrophic factor (GDNF). As used herein, “TGF-β subfamily,” “TGF-βs,” “TGF-β ligands” and grammatical equivalents thereof refer to the TGF-β subfamily members, unless specifically indicated otherwise.

The TGF-β superfamily is in turn a subset of the cysteine knot Cytokine superfamily. Additional members of the cysteine knot cytokine superfamily include, but are not limited to, platelet derived growth factor (PDGF), vascular endothelial growth factor (VEGF), placenta growth factor (PIGF), noggin, neurotrophins (BDNF, NT3, NT4, and βNGF), gonadotropin, follitropin, lutropin, interleukin-17, and coagulogen.

Publications disclosing these sequences, as well as their chemical and physical properties, include: BMP-7 and OP-2 (U.S. Pat. No. 5,011,691; U.S. Pat. No. 5,266,683; Ozkaynak et al., EMBO J., 9, pp. 2085-2093 (1990); OP-3 (WO94/10203 (PCT US93/10520)), BMP-2, BMP-4, (WO88/00205; Wozney et al. Science, 242, pp. 1528-1534 (1988)), BMP-5 and BMP-6, (Celeste et al., PNAS, 87, 9843-9847 (1991)), Vgr-1 (Lyons et al., PNAS, 86, pp. 4554-4558 (1989)); DPP (Padgett et al. Nature, 325, pp. 81-84 (1987)); Vg-1 (Weeks, Cell, 51, pp. 861-867 (1987)); BMP-9 (WO95/33830 (PCT/U595/07084); BMP-10 (WO94/26893 (PCT/US94/05290); BMP-11 (WO94/26892 (PCT/US94/05288); BMP-12 (WO95/16035 (PCT/US94/14030); BMP-13 (WO95/16035 (PCT/US94/14030); GDF-1 (WO92/00382 (PCT/US91/04096) and Lee et al. PNAS, 88, pp. 4250-4254 (1991); GDF-8 (WO94/21681 (PCT/US94/03019); GDF-9 (WO94/15966 (PCT/US94/00685); GDF-10 (WO95/10539 (PCT/US94/11440); GDF-11 (WO96/01845 (PCT/US95/08543); BMP-15 (WO96/36710 (PCT/US96/06540); MP-121 (WO96/01316 (PCT/EP95/02552); GDF-5 (CDMP-1, MP52) (WO94/15949 (PCT/U594/00657) and WO96/14335 (PCT/U594/12814) and WO93/16099 (PCT/EP93/00350)); GDF-6 (CDMP-2, BMP13) (WO95/01801 (PCT/US94/07762) and WO96/14335 and WO95/10635 (PCT/US94/14030)); GDF-7 (CDMP-3, BMP12) (WO95/10802 (PCT/US94/07799) and WO95/10635 (PCT/US94/14030)) The above publications are incorporated herein by reference.

As used herein, “TGF-β superfamily member” or “TGF-β superfamily protein,” means a protein known to those of ordinary skill in the art as a member of the Transforming Growth Factor-β (TGF-β) superfamily. Structurally, such proteins are homo or heterodimers expressed as large precursor polypeptide chains containing a hydrophobic signal sequence, an N-terminal pro region of several hundred amino acids, and a mature domain comprising a variable N-terminal region and a highly conserved C-terminal region containing approximately 100 amino acids with a characteristic cysteine motif having a conserved six or seven cysteine skeleton. These structurally-related proteins have been identified as being involved in a variety of developmental events.

The term “morphogenic protein” refers to a protein belonging to the TGF-β superfamily of proteins which has true morphogenic activity. For instance, such a protein is capable of inducing progenitor cells to proliferate and/or to initiate a cascade of events in a differentiation pathway that leads to the formation of cartilage, bone, tendon, ligament, neural or other types of differentiated tissue, depending on local environmental cues. Thus, morphogenic proteins useful in this invention can behave differently in different surroundings. In certain embodiments, a morphogenic protein of this invention can be a homodimer species or a heterodimer species.

The term “osteogenic protein (OP)” refers to a morphogenic protein that is also capable of inducing a progenitor cell to form cartilage and/or bone. The bone can be intramembranous bone or endochondral bone. Most osteogenic proteins are members of the BMP subfamily and are thus also BMPs. However, the converse can not be true. According to this invention, a BMP identified by DNA sequence homology or amino acid sequence identity must also have demonstrable osteogenic or chondrogenic activity in a functional bioassay to be an osteogenic protein. Appropriate bioassays are well known in the art; a particularly useful bioassay is the heterotopic bone formation assay (see, U.S. Pat. No. 5,011,691; U.S. Pat. No. 5,266,683, for example).

Structurally, BMPs are dimeric cysteine knot proteins. Each BMP monomer comprises multiple intramolecular disulfide bonds. An additional intermolecular disulfide bond mediates dimerization in most BMPs. BMPs may form homodimers. Some BMPs may form heterodimers. BMPs are expressed as pro-proteins comprising a long pro-domain, one or more cleavage sites, and a mature domain. The pro-domain is believed to aid in the correct folding and processing of BMPs. Furthermore, in some but not all BMPs, the pro-domain may noncovalently bind the mature domain and may act as an inhibitor (e.g., Thies et al. (2001) Growth Factors 18:251-259).

BMPs are naturally expressed as pro-proteins comprising a long pro-domain, one or more cleavage sites, and a mature domain. This pro-protein is then processed by the cellular machinery to yield a dimeric mature BMP molecule. The pro-domain is believed to aid in the correct folding and processing of BMPs. Furthermore, in some but not all BMPs, the pro-domain may noncovalently bind the mature domain and may act as a chaperone, as well as an inhibitor (e.g., Thies et. al. (2001) Growth Factors, 18:251-259).

As contemplated herein, the term “BMP” refers to a protein belonging to the BMP subfamily of the TGF-β superfamily of proteins defined on the basis of DNA homology and amino acid sequence identity. According to this invention, a protein belongs to the BMP subfamily when it has at least 50% amino acid sequence identity with a known BMP subfamily member within the conserved C-terminal cysteine-rich domain that characterizes the BMP subfamily. Members of the BMP subfamily can have less than 50% DNA or amino acid sequence identity overall. As used herein, the term “BMP” further refers to proteins which are amino acid sequence variants, domain-swapped variants, and truncations and active fragments of naturally occurring bone morphogenetic proteins, as well as heterodimeric proteins formed from two different monomeric BMP peptides, such as BMP-2/7; BMP-4/7: BMP-2/6; BMP-2/5; BMP-4/7; BMP-4/5; and BMP-4/6 heterodimers. Suitable BMP variants and heterodimers include those set forth in US 2006/0235204; WO 07/087,053; WO 05/097825; WO 00/020607; WO 00/020591; WO 00/020449; WO 05/113585; WO 95/016034 and WO93/009229.

The terms “drug,” “medicament,” or “biologic agent”/“biologic agent” (i.e., biologically active agent) as used herein include without limitation biologically, physiologically or pharmacologically active substances that can act locally or systemically in the body. A biologic agent is a substance used for the treatment, prevention, diagnosis, cure or mitigation of disease or illness, a substance which affects the structure or function of the body, or pro-drugs, which become biologically active or more active after they reside in or contact a preferred physiological environment. Also, various forms of a biologic agent can be used. These include without limitation forms such as uncharged molecules, molecular complexes, salts, ethers, esters, amides, etc., which are biologically activated when injected into the body. Preferred biologic agents include, but are not limited to, proteins having therapeutic or prophylactic activity, including enzymes, growth factors, hormones, differentiation factors, cytokines, chemokines, and antibodies.

To those skilled in the art, any biologic agent that can be released in an aqueous environment can be utilized in the disclosed invention. In a preferred embodiment, the biologic agent is proteinaceous. In another preferred embodiment, the biologic agent is minimally soluble. In a more preferred embodiment, the biologic agent is substantially physiologically insoluble. In a further preferred embodiment, the biologic agent is substantially insoluble at physiological pH. In another preferred embodiment, the biologic agent is one that can persist, after dosing, in vivo, with effectiveness for 1 hour, more preferably 24 hours, more preferably 48 hours, still more preferably one week, still more preferably one month, yet still more preferably several months. In a more particularly preferred embodiment, the biologic agent is a member of the TGF-β superfamily. In a still more particularly preferred embodiment, the biologic agent is selected from the group consisting of BMP-2, BMP-4, BMP-5, BMP-6, BMP-7, GDF-5, GDF-6, GDF-7, as well as any and all variants and homologues thereof. For instance, useful BMPs include those containing sequences, which are homologues or variants, that share at least 50%, preferably at least 60%, more preferably at least 70% and most preferably at least 85%, amino acid sequence identity with the C-terminal cysteine domain of BMP-2, BMP4, BMP-5, BMP-6, BMP-7, GDF-5, GDF-6, or GDF-7. As contemplated herein, preferred BMPs include biologically active variants of any such BMPs, including variants containing conservative amino acid substitutions. All that is required by the present invention is that these variants retain biological activity comparable to the native form. As used herein, the term “BMP related protein” or “BMP related proteins” means any one or all of the foregoing proteins.

Morphogenic proteins useful herein include any known naturally occurring native proteins, including allelic, phylogenetic counterparts and other variants thereof. These variants include forms having varying glycosylation patterns, varying N-termini, and active truncated or mutated forms of a native protein. Useful morphogenic proteins also include those that are biosynthetically produced (e.g., “muteins” or “mutant proteins”) and those that are new, morphogenically active members of the general morphogenic family of proteins.

### Modes of Administration and Delivery; Including Vascular Access Structures

Of particular importance, the present invention contemplates methods of systemic treatment using proteins, such as but not limited to those of the TGF-β superfamily, which are miniminally invasive. As used herein, “systemic” means non-local. The skilled practitioner will understand that non-local can include a method whereby a protein or other bioactive agent is introduced to a subject at a single local site, such as but not limited to a peripheral percutaneous site or a central site, so as to effectuate treatment of the subject's whole body rather than just at the single local site. As used herein, “minimally-invasive” means non-invasive or non-open-field surgical methods. The skilled practitioner will understand that minimally-invasive methods can include procedures involving an incision(s) or implantation of a medical device(s).

As already stated, the present invention is based on the discovery that a minimally-soluble bioactive agent can be provided to a subject other than by conventional routes such as oral administration, peritoneal injection, or repetitive peripheral injections. That is, a minimally-soluble bioactive agent such as a protein can now be provided effectively via a systemic route without adverse effects and without surgical intervention.

For purposes of this invention, “delivery site” means the anatomical site at which the bioactive agent actually comes into direct contact with blood; whereas, “administration site” means the anatomical site at which the bioactive agent is physically first introduced to a recipient. For example, according to one embodiment, the administration site may be the site where the catheter through which the bioactive agent is administered is introduced into the body of a recipient.

The invention exploits the discovery that certain specific physiological criteria are determinative in successful administration and delivery of a minimally-soluble bioactive agent such as a protein, including an exemplary protein such as BMP-7. In the first instance, practice of the invention requires that the intravascular site of actual delivery be substantially uncompromised. For example, the most preferred site is trauma-free; for example, edema-free. The integrity of perivascular, vascular and/or vessel intima tissue at the most preferred site is intact. Indicators of intima integrity include the extent to which protein enters or leaks into the vascular, perivascular and/or nonvascular tissue at the delivery site; no leakage or penetration of the tissue is preferred. That is, according to the teachings of the present invention, vascular, perivascular and/or non-vascular tissue at, near or adjacent the delivery site should be substantially free of biologic agent following delivery. Each of the foregoing is readily measurable by one of ordinary skill in the art using routine materials and methods.

Regarding yet another criterion, the biologic agent should disperse at a rate of at least 1 ml/min upon its delivery. Furthermore, the skilled practitioner will recognize that dispersal rate can be manipulated; a preferred dispersal rate is comparable to that which occurs at a central venous site. In short, any mode of administration and delivery which recreates and/or simulates the physiological and anatomical conditions associated with a central venous site or a deep vein site typically accessed by a central venous line or catheter is within the scope of the present invention.

Yet another criterion for practicing the present invention relates to the rate at which the protein solution is introduced to or admixes with the blood at the intravascular delivery site. The skilled practitioner will appreciate that manipulation of the rate of introduction or admixing permits the practitioner to manipulate the actual protein concentration in the solution being introduced. A slower introduction rate which effectively reduces the protein concentration at the delivery site permits higher concentrations of starting materials which results in lessened administered volumes as well as lessened precipitation and inadvertent penetration of the nonvascular tissue by the administered agent. Each of the foregoing indicia is readily measurable by one of ordinary skill in the art using routine materials and methods.

Thus, in a currently preferred embodiment of the invention, a method of treatment of an injured or diseased tissue comprises administering to an administration site a composition comprising a biologic agent and delivering to an intravascular delivery site the composition such that intima tissue integrity at the delivery site is substantially uncompromised. In this embodiment, the biologic agent disperses from the delivery site at a rate and in an amount effective to treat the injured or diseased tissue. In certain embodiments, the administration site and the delivery site are the same. In a particularly preferred embodiment, the delivery site is venular-valve-free. In another, the blood flow rate at the delivery site is sufficient to provide a biologically effective dose at a site remote from the site of delivery. In yet another, the delivering step is accomplished using an intravascular apparatus having a distal end with a non-damaging configuration. In one such embodiment, the apparatus is an indwelling intravascular catheter with a non-damaging configuration.

In a related aspect, the present invention is directed to a composition comprising a biologic agent and a vascular access structure. As contemplated herein, a vascular access structure is any device or apparatus which can be used to provide an effective amount of biologic agent in accordance with the present invention. A most preferred vascular access structure is one which does not disrupt intima tissue at the site of delivery. In accordance with the present invention, a vascular access structure is a device or apparatus which provides access to a subject's vasculature; the vasculature can be either central or peripheral. As contemplated by the present invention, a vascular access structure is implantable on the exterior or the interior of a subject. In certain embodiments, such a structure can operate to deliver a biologic agent into the subject's blood stream at a site remote from the implantation site. A central delivery site is preferable.

The skilled practitioner will be familiar with vascular access structures generally, the most common of which are described briefly herein; their utility with the present invention will be readily apparent. Structures contemplated herein include, for example, a Hickman line, a Peripherally Inserted Central Catheter (PICC) which is an exemplary central venous catheter, and a portacath. There are several types of central venous catheters. As described in more detail below, a PICC line typically is inserted into a vein in the arm rather than a vein in the neck (internal jugular vein) or chest (subclavian vein) or groin (femoral vein). The tunneled catheter is another type which is surgically inserted into a vein in the neck or chest and passed under the skin. Only the end of the catheter is brought through the skin. Additionally, there is the type known as the implanted port or vascular access port. This, too, is tunneled but the entire device, including the port which receives the liquid medication, is typically implanted subcutaneously.

The PICC typically extends from an arm vein into the superior vena cava near the heart and typically provides central IV access for several weeks up to several months where they are placed in a way that the tip of the catheter remains in a relatively large vein, but do not extend into the largest central vein, they are known as midline catheters. Non-tunneled central catheters are larger caliber than peripherally inserted central catheters and are designed to be placed via a more central vein such as the jugular vein in the neck or the femoral vein in the groin. The tunneled catheter has a cuff that stimulates tissue growth that will help hold it in place in the body. Examples of the tunneled catheter include HICKMAN® catheters, BROVIAC® catheters and GROSHONG® catheters. HICKMAN®, BROVIAC® and/or GROSHONG® are registered trademarks of C. R. Bard, Inc. and its related company, BCR, Inc. The tunneled catheter is preferred when access to the vein is needed for long period of time. The port catheter, or subcutaneous implantable port, is a permanent device that consists of a catheter attached to a small reservoir, both of which are placed under the skin similar to tunnel catheters. An open port catheter is similar but remains on top of the skin.

In certain currently preferred embodiments, a method of treatment of an injured or diseased tissue comprises the step of providing to an administration site a composition comprising a biologic agent and a vascular access structure, whereupon the biologic agent is delivered in an amount effective to treat the injured or diseased tissue. Preferably, the physical administration site is remote from the actual site of delivery of the biologic agent. In certain embodiments, the delivery site is a non-peripheral site. For example, in one embodiment, the delivery site is a central site. In certain embodiments, the administration site is a peripheral site.

In keeping with the teachings of the present invention, the currently preferred biologic agent is BMP-7 and injured or diseased non-mineralized tissue is the currently preferred object of treatment. Such injured or diseased tissue can be an organ. In a particularly preferred method of treatment, the biologic agent is bioavailable for at least about 0.5 hours, more preferably at least about 2 hours; at least about 8 hours; for about 1 day, preferably more than 1 day. And, an effective amount is about 10 microgram to about 1000 microgram of biologic agent, more preferably about 50 microgram to about 500 microgram, most preferably about 100 microgram to 300 microgram biologic agent per kg of patient body weight.

Preferably, in one embodiment of the present invention, a vascular line is placed such that it is easily accessible and the catheter access has sufficiently healed into place; for example, a vascular access port at the injection end of the catheter is which the therapeutic agent is introduced. The vascular line can be placed with or without surgery and then be allowed to heal to minimize or avoid any leakage at the puncture site into the blood vessel. According to the invention, the term “heal” or “healed” suggests that tissue integrity is substantially uncompromised and/or that the venipuncture site is substantially free of tissue damage. In other words, the term “heal” does not require complete repair of the injured or compromised site, although a tissue that is “healed” may be completely repaired or uncompromised. The term “heal” or “healed,” in one embodiment, suggests that damaged or diseased tissue has been substantially replaced with new tissue growth, which may include scar tissue.

The skilled artisan will appreciate that the treatment and administration methods of the present invention can be modified or varied to optimize treatment of an individual in view of numerous factors including, but not limited to, the indication, the pathology of the disease, and the physical characteristics of the individual.

### Therapeutic Interventions

As explained above, the invention also provides methods of treatment using a composition of the present invention containing any biologic agent, or formulation thereof, in an amount effective to ameliorate and/or prevent any known or potential condition for which the biologic agent is efficacious. As used herein “an effective amount” means an amount of a biologic agent that is effective to treat a condition in a living organism to which it is administered. For example, the BMP formulations of the invention can be used to treat patients suffering from disease or injury of connective tissues, such as bone and cartilage. Additionally, as described below, the BMP formulations of the invention can be used to treat diseases or injuries of other tissues.

In one embodiment of the invention, the injury to be ameliorated is a mineralized or non-mineralized skeletal tissue injury. In another embodiment, the injury or disease to be ameliorated is metabolic bone disease, osteoarthritis, osteochondral disease, rheumatoid arthritis, osteoporosis, Paget's disease, periodontitis, dentinogenesis, chondral disease, trauma-induced and inflammation-induced cartilage degeneration, age-related cartilage degeneration, articular cartilage injuries and diseases, full thickness cartilage diseases, superficial cartilage defects, sequelae of systemic lupus erythematosis, sequelae of scleroderma, periodontal tissue regeneration, herniation and rupture of intervertebral discs, degenerative diseases of the intervertebral disc, osteocondrosis, or injuries and diseases of ligament, tendon, synovial capsule, synovial membrane and meniscal tissues. In another embodiment, the injury or disease to be ameliorated is liver disease, liver resection, hepatectomy, renal disease, chronic renal failure, central nervous system ischemia or trauma, neuropathy, motor neuron injury, dendritic cell deficiencies and abnormalities, Parkinson's disease, ophthalmic disease, ocular scarring, retinal scarring, or ulcerative diseases of the gastrointestinal tract.

BMPs are capable of inducing the developmental cascade of bone morphogenesis and tissue morphogenesis for a variety of tissues in mammals different from bone or cartilage. This morphogenic activity includes the ability to induce proliferation and differentiation of progenitor cells, and the ability to support and maintain the differentiated phenotype through the progression of events that results in the formation of bone, cartilage, non-mineralized skeletal or connective tissues, and other adult tissues.

For example, BMPs can be used for treatment to prevent loss of and/or increase bone mass in metabolic bone diseases. General methods for treatment to prevent loss of and/or increase bone mass in metabolic bone diseases using osteogenic proteins are disclosed in U.S. Pat. No. 5,674,844, the disclosures of which are hereby incorporated by reference. BMPs of the present invention can be used for periodontal tissue regeneration. General methods for periodontal tissue regeneration using osteogenic proteins are disclosed in U.S. Pat. No. 5,733,878, the disclosures of which are hereby incorporated by reference. BMPs can be used for liver regeneration. General methods for liver regeneration using osteogenic proteins are disclosed in U.S. Pat. No. 5,849,686, the disclosures of which are hereby incorporated by reference. BMPs can be used for treatment of chronic renal failure. General methods for treatment of chronic renal failure using osteogenic proteins are disclosed in U.S. Pat. No. 6,861,404, the disclosures of which are hereby incorporated by reference. BMPs can be used for enhancing functional recovery following central nervous system ischemia or trauma. General methods for enhancing functional recovery following central nervous system ischemia or trauma using osteogenic proteins are disclosed in U.S. Pat. No. 6,407,060, the disclosures of which are hereby incorporated by reference. BMPs can be used for inducing dendritic growth. General methods for inducing dendritic growth using osteogenic proteins are disclosed in U.S. Pat. No. 6,949,505, the disclosures of which are hereby incorporated by reference. BMPs can be used for inducing neural cell adhesion. General methods for inducing neural cell adhesion using osteogenic proteins are disclosed in U.S. Pat. No. 6,800,603, the disclosures of which are hereby incorporated by reference. BMPs can be used for treatment and prevention of Parkinson's disease. General methods for treatment and prevention of Parkinson's disease using osteogenic proteins are disclosed in U.S. Pat. No. 6,506,729, the disclosures of which are hereby incorporated by reference.

Additionally, BMPs can be used to repair diseased or damaged mammalian tissue. The existing tissue at the locus, whether diseased or damaged, provides the appropriate matrix to allow the proliferation and tissue-specific differentiation of progenitor cells. In addition, a damaged or diseased tissue locus, particularly one that has been further assaulted by surgical means, provides a morphogenically permissive environment.

BMPs also can be used to prevent or substantially inhibit scar tissue formation following an injury. It can induce tissue morphogenesis at the locus, preventing the aggregation of migrating fibroblasts into non-differentiated connective tissue. For example, BMPs can be used for protein-induced morphogenesis of substantially injured liver tissue following a partial hepatectomy.

As another example, BMPs can also be used to induce dentinogenesis. To date, the unpredictable response of dental pulp tissue to injury is a basic clinical problem in dentistry. As yet another example, BMPs can induce regenerative effects on central nervous system (CNS) repair can be assessed using a rat brain stab model.

In the case of skeletal disorders, a number of factors can cause or contribute to cartilage degeneration in mammals, including trauma and inflammatory disease. Damage to cells resulting from the effects of inflammatory response has been implicated as the cause of reduced cartilage function or loss of cartilage function in diseases of the joints (e.g., rheumatoid arthritis (RA) and osteoarthritis (OA)). In addition, autoimmune diseases such as systemic lupus erythematosis (SLE) and scleroderma can also be characterized by a degradation of connective tissue. In the case of some cartilage degenerative diseases such as osteoarthritis (OA), the mechanisms that turn the normal aging of articular cartilage into the pathological OA process are currently unknown. Each of the foregoing diseases can be effectively treated with the materials and methods of the present invention.

As stated earlier, the BMP formulations of the invention can be used effectively to treat skeletal diseases or injuries. For example, the BMP formulations of the invention can be used to treat a disease or injury resulting in cartilage degradation or a cartilage defect, such as a degenerative intervertebral disc, or other fibrocartilaginous tissue, including a tendon, a ligament or a meniscus. The formulations of the invention can also be used to treat a defect or degeneration of articular cartilage, as set forth in published PCT application WO 05/115438, such as the cartilage lining of a joint, such as a synovial joint, including a knee, an elbow, a hip, or a shoulder. In another embodiment, the formulations of the invention are used to treat an articular cartilage defect site, such as a chondral defect or an osteochondral defect, in a joint. Such articular cartilage defects can be the result of a disease process, such as osteoarthritis or rheumatoid arthritis, or due to injury of the joint.

### Formulations

Biologic agents, and especially BMPs, of the present invention can be formulated for administration to a mammal, preferably a human, in need thereof as part of a pharmaceutical composition. In certain embodiments, the biologic agent can be administered in or with an appropriate carrier or bulking agent including, but not limited to, biocompatible oil such as sesame oil, hyaluronic acid, cyclodextrins, lactose, raffinose, mannitol, carboxy methyl cellulose, thermo or chemo-responsive gels, sucrose acetate isobutyrate. The skilled artisan would understand that a bulking agent or carrier can facilitate the delivery of the condensed dosage forms of the biologic agents disclosed herein wherein the dosage volumes include, but are not limited to, volumes of 20 μl or less, for example. In a particularly preferred embodiment of the present invention, a bulking agent can be used in conjunction with a biologic agent of the present invention that is substantially insoluble at physiological pH, to increase the dissolution of the biologic agent such that the bulking agent acts classically as a carrier to release of the biologic agent. In a still more particularly preferred embodiment, the biologic agent is BMP-7. A currently preferred embodiment of the present invention comprises a BMP formulation comprising trehalose, preferably trehalose in a lacate buffer, most preferably BMP-7 in a buffer of 10 mM lactate comprising 9% trehalose. It is within the skill in the art to practice the aforementioned embodiments of the present invention, as well as any and all variants and modifications of the present invention that the skilled artisan would recognize provide effective dosing of the biologic agent in vivo.

Still further, the biologic agent of the present invention can be administered to the mammal in need thereof either alone or in combination with another substance known to have a beneficial effect on tissue morphogenesis. Examples of such substances (herein, cofactors) include without limitation substances that promote tissue repair and regeneration and/or inhibit inflammation. Examples of useful cofactors for stimulating bone tissue growth in osteoporotic individuals, for example, include but are not limited to, vitamin D3, calcitonin, prostaglandins, parathyroid hormone, dexamethasone, estrogen and IGF-I or IGF-II. Useful cofactors for nerve tissue repair and regeneration can include, but are not limited to, nerve growth factors. Other useful cofactors include symptom-alleviating cofactors, including, but not limited to, antiseptics, antibiotics, antiviral and antifungal agents, analgesics and anesthetics.

As will be appreciated by those skilled in the art, the concentration of the compounds described in a therapeutic composition will vary depending upon a number of factors, including without limitation the dosage of the drug to be administered and the chemical characteristics (e.g., hydrophobicity) of the compounds employed. The preferred dosage is likely to depend on variables including, but not limited to, the type and extent of a disease, tissue loss or defect, the overall health status of the particular patient, the relative biological efficacy of the compound selected, the formulation of the compound, and the presence and types of excipients in the formulation. The therapeutic molecules of the present invention may be provided to an individual where typical doses range from about 10 ng/kg to about 1 g/kg of body weight per day; with a preferred dose range being from about 0.1 mg/kg to 100 mg/kg of body weight, and with a more particularly preferred dosage range of 10-1000 μg/dose. The skilled clinician would appreciate that the effective doses of the present invention can be modified in light of numerous factors including, but not limited to, the indication, the pathology of the disease, and the physical characteristics of the individual. It is also clearly within the skill in the art to vary, modify, or optimize doses in view of any or all of the aforementioned factors.

Pursuant to the parameters and conditions of certain embodiments of the invention, the availability of the biologic agent can be controlled. In particular, the rate and extent of availability of the biologic agent from a formulation can be controlled by variation of properties such as but not limited to polymer type and molecular weight, use of a rate modifying agent, use of plasticizers and leachable agents and the concentrations and kinds of thermoplastic polymer and biologic agent.

Rate modifying agents, plasticizers and leachable agents can be included to manage the rate of release of biologic agent and the pliability of a matrix in which it is optionally contained. The rate modifying agent can increase or retard the rate of release depending upon the nature of the rate modifying agent incorporated into a matrix. Known plasticizers as well as organic compounds that are suitable for secondary pseudobonding in polymer systems are acceptable as rate modifying agents and also as pliability modifiers and leaching agents. Generally these agents are esters of mono, di and tricarboxylic acids, diols and polyols, polyethers, non-ionic surfactants, fatty acids, fatty acid esters, oils such as vegetable oils, and the like. The concentrations of such agents within the matrix can range in amount up to 60 wt % relative to the total weight of the matrix, preferably up to 30 wt % and more preferably up to 15 wt %. Generally, these rate modifying agents, leaching agents, plasticizers and pliability modifiers and their application are described in U.S. Pat. Nos. 5,702,716 and 5,447,725, the disclosures of which are incorporated herein by reference with the proviso that the polymers to be used are biocompatible and/or biodegradable. The skilled artisan would appreciate that the present invention comprises any and all agents within the art that can increase the solubilization rate of the biologic agent or the degradation rate or erosion rate of any carrier for the biologic agent. Hence, other agents amenable to the practice of the present invention include, but are not limited to, co-localized pH modifying agents and tonicity modifiers. In a particularly preferred embodiment, the composition of the present invention comprises a co-localized pH modifying agent or tonicity modifier provided in a concentration or quantity that substantially increases the solubilization rate of the biologic agent. In another preferred embodiment, the composition of the present invention comprises a co-localized pH modifying agent or tonicity modifier provided in a concentration or quantity that substantially increases the degradation rate or erosion rate of the carrier. The skilled artisan would appreciate that the rate modifying agents, leaching agents, plasticizers, pliability modifiers, pH modifying agents, and tonicity modifiers of the present invention can be substituted, modified, varied in nature or concentration, and optimized in view of numerous factors, including, but not limited to, the desired release rate, the nature of the carrier (if any), the indication, the pathology of the disease, and the physical characteristics of the individual.

Formulations of biologic agents of this invention can further include one or more excipients. Examples of excipients are described in the Handbook of Pharmaceutical Excipients, published jointly by the American Pharmaceutical Association and the Pharmaceutical Society of Great Britain. Excipients that can be employed in the making and use of the formulations and pharmaceutical compositions of the present invention include, but are not limited to; acidifying agents, such as, acetic acid, glacial acetic acid, citric acid, fumaric acid, hydrochloric acid, diluted hydrochloric acid, malic acid, nitric acid, phosphoric acid, diluted phosphoric acid, sulfuric acid, tartaric acid; alcohol denaturants, such as, denatonium benzoate, methyl isobutyl ketone, sucrose octacetate; alkalizing agents, such as, strong ammonia solution, ammonium carbonate, diethanolamine, diisopropanolamine, potassium hydroxide, sodium bicarbonate, sodium borate, sodium carbonate, sodium hydroxide, trolamine; antifoaming agents, such as, dimethicone, simethicone; antimicrobial preservatives, such as, benzalkonium chloride, benzalkonium chloride solution, benzelthonium chloride, benzoic acid, benzyl alcohol, butylparaben, cetylpyridinium chloride, chlorobutanol, chlorocresol, cresol, dehydroacetic acid, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, sorbic acid, thimerosal, thymol; antioxidants, such as, ascorbic acid, acorbyl palmitate, butylated hydroxyanisole, butylated hydroxytoluene, hypophosphorous acid, monothioglycerol, propyl gallate, sodium formaldehyde sulfoxylate, sodium metabisulfite, sodium thiosulfate, sulfur dioxide, tocopherol, tocopherols excipients; buffering agents, such as, acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate; chelating agents, such as, edetate disodium, ethylenediaminetetraacetic acid and salts, edetic acid; coating agents, such as, sodium carboxymethylcellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyethylene glycol, polyvinyl acetate phthalate, shellac, sucrose, titanium dioxide, carnauba wax, microcystalline wax, zein; colors, such as, caramel, red, yellow, black or blends, ferric oxide; complexing agents, such as, ethylenediaminetetraacetic acid and salts (EDTA), edetic acid, gentisic acid ethanolmaide, oxyquinoline sulfate; dessicants, such as, calcium chloride, calcium sulfate, silicon dioxide; emulsifying and/or solubilizing agents, such as, acacia, cholesterol, diethanolamine (adjunct), glyceryl monostearate, lanolin alcohols, lecithin, mono- and di-glycerides, monoethanolamine (adjunct), oleic acid (adjunct), oleyl alcohol (stabilizer), poloxamer, polyoxyethylene 50 stearate, polyoxyl 35 caster oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan monolaurate, soritan monooleate, sorbitan monopalmitate, sorbitan monostearate, stearic acid, trolamine, emulsifying wax; filtering aids, such as, powdered cellulose, purified siliceous earth; glidants and/or anticaking agents, such as, calcium silicate, magnesium silicate, colloidal silicon dioxide, talc; humectants, such as, glycerin, hexylene glycol, propylene glycol, sorbitol; plasticizers, such as, castor oil, diacetylated monoglycerides, diethyl phthalate, glycerin, mono- and di-acetylated monoglycerides, polyethylene glycol, propylene glycol, triacetin, triethyl citrate; polymer membranes, such as, cellulose acetate; solvents, such as, acetone, acetic acid, alcohol, diluted alcohol, amylene hydrate, benzyl benzoate, butyl alcohol, carbon tetrachloride, chloroform, corn oil, cottonseed oil, ethyl acetate, glycerin, hexylene glycol, isopropyl alcohol, methyl alcohol, methylene chloride, methyl isobutyl ketone, mineral oil, peanut oil, polyethylene glycol, propylene carbonate, propylene glycol, sesame oil, water for injection, sterile water for injection, sterile water for irrigation, purified water; sorbents, such as, powdered cellulose, charcoal, purified siliceous earth, and carbon dioxide sorbents; stiffening agents, such as, hydrogenated castor oil, cetostearyl alcohol, cetyl alcohol, cetyl esters wax, hard fat, paraffin, polyethylene excipient, stearyl alcohol, emulsifying wax, white wax, yellow wax; suspending and/or viscosity-increasing agents, such as, acacia, agar, alginic acid, aluminum monostearate, bentonite, purified bentonite, magma bentonite, carbomer 934p, carboxymethylcellulose calcium, carboxymethylcellulose sodium, carboxymethycellulose sodium 12, carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, propylene glycol alginate, silicon dioxide, colloidal silicon dioxide, sodium alginate, tragacanth, xanthan gum; and wetting and/or solubilizing agents, such as, benzalkonium chloride, benzethonium chloride, cetylpyridinium chloride, docusate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20, cetostearyl ether, polyoxyl 40 stearate, polysorbate 20, polysorbate 40, polysorbate 60, polysorbate 80, sodium lauryl sulfate, sorbitan monolaureate, sorbitan monooleate, sorbitan monopalmitate, sorbitan monostearate, tyloxapol.

### Bioactive Co-Agents

The present invention also contemplates “bioactive co-agents” that can be co-administered with the biologic agent compositions of the present invention include, but are not limited to, anabolic agents, antacids, anti-asthmatic agents, anti-cholesterolemic and anti-lipid agents, anti-coagulants, anti-convulsants, anti-diarrheals, anti-emetics, anti-infective agents including, for example, antibacterial and antimicrobial agents, anti-inflammatory agents, anti-manic agents, antimetabolite agents, anti-nauseants, anti-neoplastic agents, anti-bone resorption agents, anti-obesity agents, anti-pyretic and analgesic agents, anti-spasmodic agents, anti-thrombotic agents, anti-tussive agents, anti-uricemic agents, anti-anginal agents, antihistamines, appetite suppressants, biologicals, cerebral dilators, coronary dilators, bronchodilators, cytotoxic agents, decongestants, diuretics, diagnostic agents, erythropoietic agents, expectorants, gastrointestinal sedatives, hyperglycemic agents, hypnotics, hypoglycemic agents, immunomodulating agents, ion exchange resins, laxatives, mineral supplements, mucolytic agents, neuromuscular drugs, peripheral vasodilators, psychotropics, sedatives, stimulants, thyroid and anti-thyroid agents, tissue growth agents, uterine relaxants, vitamins, or antigenic materials.

More particularly, the bioactive co-agents preferred for co-administration with the present invention include, but are not limited to, androgen inhibitors, polysaccharides, growth factors, hormones, bisphosphonates, anti-angiogenesis factors, dextromethorphan, dextromethorphan hydrobromide, noscapine, carbetapentane citrate, chlophedianol hydrochloride, chlorpheniramine maleate, phenindamine tartrate, pyrilamine maleate, doxylamine succinate, phenyltoloxamine citrate, phenylephrine hydrochloride, phenylpropanolamine hydrochloride, pseudoephedrine hydrochloride, ephedrine, codeine phosphate, codeine sulfate morphine, mineral supplements, cholestryramine, N-acetylprocainamide, acetaminophen, aspirin, ibuprofen, phenyl propanolamine hydrochloride, caffeine, guaifenesin, aluminum hydroxide, magnesium hydroxide, peptides, polypeptides, proteins, amino acids, hormones, interferons, cytokines, and vaccines. Other representative bioactive co-agents that can be co-administered with the present invention include, but are not limited to, peptide drugs, protein drugs, desensitizing materials, antigens, anti-infective agents such as antibiotics, antimicrobial agents, antiviral, antibacterial, antiparasitic, antifungal substances and combination thereof, antiallergenics, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, cardioactive agents, nonsteroidal anti-inflammatory agents, antiparkinsonian agents, antihypertensive agents, .beta.-adrenergic blocking agents, nutritional agents, and the benzophenanthridine alkaloids. The bioactive co-agent may further be a substance capable of acting as a stimulant, sedative, hypnotic, analgesic, anticonvulsant, and the like.

The bioactive co-agent may also be a substance, or metabolic precursor thereof, which is capable of promoting growth and survival of cells and tissues, or augmenting the activity of functioning cells, as for example, blood cells, neurons, muscle, bone marrow, bone cells and tissues, and the like. For example, bioactive co-agents that may be co-administered include without limitation a nerve growth promoting substance, as for example, a ganglioside, phosphatidylserine, a nerve growth factor, brain-derived neurotrophic factor. The bioactive co-agent may also be a growth factor for soft or fibrous connective tissue as, for example, a fibroblast growth factor, an epidermal growth factor, an endothelial cell growth factor, a platelet derived growth factor, an insulin-like growth factor, a periodontal ligament cell growth factor, to name but a few.

### Examples

### 1. Minimally-Invasive Systemic Delivery of an Exemplary BMP

Central Venous Delivery of BMP-7 in a Rodent

(a) Rat Study No. 1: Determination of Maximum Tolerated Dose and 28-Day Feasibility Toxicity Study of BMP-7 Administered Intravenously to Female Sprague-Dawley Rats via Intravenous Catheters

The purpose of this study was to determine a maximum tolerated dose (MTD) of the exemplary BMP, BMP-7. Four female Sprague-Dawley rats were dosed via jugular vein vascular access ports (VAPs) with either 10 mg/kg of BMP-7 (n=2) as a 1 mg/mL solution or an equal volume (1 mL) of 10 mM Lactate/9% Trehalose buffer (n=2) 5 days a week for 4 weeks for a total of 20 doses in 28 days. Blood was collected for analysis of serum anti-BMP-7 antibodies before exposure and at day 28 after dosing. Animals were evaluated by assessment of clinical signs, body weights, CBC, serum chemistry, necropsy, and organ weights. Serum samples were analyzed to determine the amount of anti-BMP-7 antibodies.

No clinical signs, altered body weights, CBC, serum chemistry, organ weights, macroscopic or microscopic findings were considered to be associated with IV administration of BMP-7. At necropsy, all organs appeared within the range of normal. The catheter of one treatment animal was clogged, and this animal had high numbers of WBC s, indicating possible bacterial contamination of the catheter.

Under the conditions of this study, a MTD was not achieved; the animals tolerated well 10 mg/kg of BMP-7. This was the MFD (maximum feasible dose) based on the BMP-7 solutions tested.

(b) Rat Study No. 2: A 28-Day Safety Evaluation of Intravenously Administered BMP-7 in Sprague-Dawley Rats

The purpose of this study was to assess the effects of 1 mg/kg BMP-7 administered intravenously (IV) via jugular catheters to female Sprague-Dawley (SD) rats. Five female SD rats were injected 6 times IV with 1 mg/kg BMP-7 via a jugular vein catheter over the course of 10 days. Five control female SD rats were injected with vehicle only IV 6 times over 10 days. Animals were assessed for changes in clinical signs before and after injections. No clinical signs were noted associated with IV administration of BMP-7 or vehicle. The IV administration of BMP-7 was associated with no signs of toxicity in rats and was well-tolerated.

Thus, these observations confirm that practice of the methods of the present invention reduce or eliminate the adverse effects typically associated with systemic treatment using a BMP.

### Central Venous Delivery of BMP-7 in Cats

(a) Pharmacokinetic profile of BMP-7 Administered Intravenously to Cats.

The purpose of this study was to evaluate any effects of BMP-7 administered IV via central venous catheters (implanted vascular access ports into a vein) on cats. Ten adult male domestic short hair cats were administered 0.3 mg/kg BMP-7 once per week for four weeks and then 1 mg/kg BMP-7 once per week for 2 weeks followed by 2 mg/kg BMP-7 once per week for 2 weeks. Cats were evaluated by clinical observations, physical examinations, clinical pathology, and body weights. All of the cats remained clinically normal throughout the study and had no signs or evidence of any adverse effects; no signs of any vascular irritation or inflammation (including no signs of any bone or cartilage formation) were observed in any cat over the course of the study. Under the conditions of this study, administration of up to 2 mg/kg BMP-7 IV via central venous catheter in male cats has no adverse findings.

### Central Venous Delivery of BMP-7 in Monkeys

(a) BMP-7 Intravenous Toxicity Study in Cynomolgus Monkeys

The objective of this study was to determine the potential toxicity of BMP-7 after 14 every-other-day intravenous (IV) injections over 4 weeks in male and female cynomolgus monkeys and to assess recovery of potential effects after a 2-week recovery period. This study consisted of 5 groups each with 3 male and 3 female cynomolgus monkeys (main study) and an additional 2 male and 2 female cynomolgus monkeys in Groups 1 and 4 to assess recovery. All animals were injected via a central venous catheter with a vascular access port. Group 1 was injected with control article (vehicle, 5 mM lactose/9% trehalose), Group 2 with 0.1 mg/kg SF BMP-7, Group 3 with 0.3 mg/kg SF BMP-7, Group 4 with 1 mg/kg SF BMP-7, and Group 5 was intended to receive 0.3 mg/kg 37 C BMP-7. Toxicity was evaluated by monitoring clinical observations, body weight, food consumption, physical and ophthalmologic examinations, body temperatures, respiratory rates, indirect blood pressure, electrocardiography (ECGs), and clinical pathology parameters (CBCs, coagulation, serum chemistry, and urinalysis), and necropsy with organ weights and microscopic evaluation of tissues.

On Day 1, animals in Group 5 received BMP-7, at a dose of approximately 1 mg/kg instead of BMP-7 at a dose of 0.3 mg/kg. Dosing was suspended for the Group 5 animals after Day 5, and the animals were removed from study.

No BMP-7—related clinical observations or changes in body weight, food consumption, ophthalmologic examinations, body temperature, respiratory rate, blood pressure, ECGs, or clinical pathology parameters were observed. The veins used for drug administration by implanted central venous line had no observed gross or microscopic findings associated with administration of BMP-7.

Animals treated with 0.1 (Group 2), 0.3 (Group 3), or 1 (Group 4) mg/kg/day of SF BMP-7 IV every other day over 28 days (for 14 doses) had no observations or findings that were considered toxicologically meaningful or clinically relevant compared with control animals (Group 1). Under the conditions of this study, the no observed adverse effect level (NOAEL) and the no-observed effect level (NOEL) were determined to be in excess of the highest dose tested (1 mg/kg BMP-7) for male and female cynomolgus monkeys.

### 2. Minimally-Invasive Systemic Delivery of an Exemplary BMP

Central Venous Delivery of BMP-7 in a Primate

(a) Primate Study No. 1

Three each of female and male Cynomolgus monkeys (Macaca fascicularis) were surgically instrumented with a vascular access port through which BMP-7 was administered and delivered according to the following protocol:

### Primate Study 1

| | | Dose | Dose | Dose | | |
|---|---|---|---|---|---|---|
| Group | | Level | Conc. | Volume | Dosing | Necropsy |
| No. | N | (mg/kg/day) | (mg/mL) | (mL/kg) | Regimen^{a,b} | Day |
| 1 | 6 | 0 | 0 | 1.0 | Once every | Day 28 |
| 2 | | 0.1 | 1.0 | 0.1 | other day from | |
| 3 | | 0.3 | | 0.3 | Days 1 to 27 | |
| 4 | | 1.0 | | 1.0 | | |

^{a}Doses for Groups 1 and 4 were administered by an approximate 30-minute infusion.
^{b}Doses for Groups 2 and 3 were administered by an approximate 15-minute infusion.

Blood serum was collected to measure BMP-7 antibody levels in control and treated subjects. On Day 28, subjects were euthanized and tissues collected for pathology evaluation both macroscopically and microscopically.

Macroscopic and microscopic findings at the administration site (i.e., lumen of the vena cava) in both control and treated subjects showed only expected signs of implantation of a chronic indwelling intravascular catheter. That is, neither control nor treated subjects show any other effects such as fibrosis or bone formation. Also, BMP-7 treated subjects showed no signs of localized vessel irritation when compared with control subjects. Thus, these data confirm that systemic administration of a BMP in accordance with the present invention obviates any adverse effects associated with conventional methods of systemic delivery of a BMP.

(b) Primate Study No. 2

Eight young adult male Cynomolgus monkeys (Macaca fascicularis) will be surgically instrumented with an iliac-based vascular access port and catheter through which BMP-7 will be administered and delivered according to the following protocol:

### Primate Study 2

| | | | | Dosage | Dose | Dosage | | Last |
|---|---|---|---|---|---|---|---|---|
| Phase | Group | | Biologic | Level | Concentration | Volume | Dosing | Day |
| No. | No. | N | Agent | (mg/kg/day) | (mg/ml) | (ml/kg) | Regimen | of Test |
| I | 1 | 2 | BMP-7 | 1.0 | 1.0 | 1.0 | Once daily | 15 |
| | | | | | | | Days 1-14 | |
| I | 2 | 2 | BMP-7 | 1.0 | 1.0 | 1.0 | Once every | 28 |
| | | | | | | | other day | |
| | | | | | | | Days 1-27 | |
| II | 3 | 2 | BMP-7 | 0.5 | 1.0 | 0.5 | Once daily | 15 |
| | | | | | | | Days 1-14 | |
| II | 4 | 2 | BMP-7 | 0.5 | 1.0 | 0.5 | Once every | 28 |
| | | | | | | | other day | |
| | | | | | | | Days 1-27 | |

As described in the study protocols, BMP-7 will be administered via the vascular access port for 14 (daily) or 27 (every other day) days. BMP-7 administration will be preceded and followed by a 3 ml flush of 10 mM lactate, 9% trehalose solution through the catheter. BMP-7 will be administered at approximately the same time each day. Blood samples will be collected from a peripheral vessel and tested for various hematological, chemical, antibody and toxicokinetic parameters. Upon completion of the study, animals will be sacrificed and a complete gross necropsy conducted including examination of various external and internal tissues and organs, further including the sites of administration and delivery.

In brief, analysis of blood samples will confirm biologically significant levels of BMP-7 were present and sufficient to induce BMP-7 circulating antibodies. In brief, post-mortem analysis will confirm no adverse effects of systemic BMP-7 administration in accordance with the present invention. Thus, this study will demonstrate that minimally-invasive systemic delivery of an exemplary BMP can be accomplished using a vascular access structure, thereby permitting sustained systemic treatment without adverse side effects.

(c) Primate Study No. 3

Four young adult male Cynomolgus monkeys (Macaca fascicularis) are surgically instrumented with an iliac-based vascular access port and catheter through which BMP-7 is administered and delivered according to the following protocol:

### Primate Study 3

| | | | | Dosage | Dosage | | Last |
|---|---|---|---|---|---|---|---|
| Group | | Biologic | Dosage | Concentration | Volume | Dosing | Day of |
| No. | N | Agent | (mg/kg/day) | (mg/ml) | (ml/kg) | Regimen | Test |
| 1 | 2 | BMP-7 | 0.5 | 1.6 | 0.31 | Once Daily | 29 |
| | | | | | | Days 1-28 | |
| 2 | 2 | BMP-7 | 1.5 | 1.6 | 0.94 | Once Daily | 29 |
| | | | | | | Days 1-28 | |

BMP-7 will be administered by introducing the prescribed volume to the port opening over about a 30 minute dosing period. As described in the study protocols, BMP-7 will be administered via the vascular access port for 28 (daily) days. BMP-7 administration will be preceded and followed by a 3 ml flush of 10 mM lactate, 9% trehalose solution through the catheter. BMP-7 will be administered at approximately the same time each day. Blood samples will be collected from a peripheral vessel and tested for various hematological, chemical, antibody and toxicokinetic parameters. Upon completion of the study, animals will be sacrificed and a complete gross necropsy conducted including examination of various external and internal tissues and organs, further including the sites of administration and delivery.

In brief, analysis of blood samples will confirm biologically significant levels of BMP-7 were present and sufficient to induce BMP-7 circulating antibodies. In brief, post-mortem analysis will confirm no adverse effects of systemic BMP-7 administration in accordance with the present invention. Thus, this study will demonstrate that minimally-invasive systemic delivery of an exemplary BMP can be accomplished using a vascular access structure, thereby permitting sustained systemic treatment without adverse side effects.

### 3. Therapeutic Uses for Minimally-Invasive Systemic Delivery of an Exemplary BMP in Humans:

(a) Osteoporosis

A population of human patients with a confirmed clinical diagnosis of osteoporosis will be administered a dose of 0.01-3.0 μg/kg of BMP-7 once weekly via a centrally located catheter in accordance with the methods of the present invention. It is expected that such treatment will modulate the disease to a statistically significant extent in the treated patient population.

(b) Metabolic Bone Disease

A population of human patients with a confirmed clinical diagnosis of metabolic bone disease will be administered a dose of 0.01-3.0 μg/kg of BMP-7 once weekly via a centrally located catheter in accordance with the methods of the present invention. It is expected that such treatment will modulate the disease to a statistically significant extent in the treated patient population.

(c) Fibrosis Including Hepatic, Pulmonary, Cardiac and Renal Manifestations

Populations of human patients with a confirmed clinical diagnosis of fibrosis including each of hepatic, pulmonary, cardiac and renal fibrosis will be administered a dose of 0.01-3.0 μg/kg of BMP-7 once weekly via a centrally located catheter in accordance with the methods of the present invention. It is expected that such treatment will modulate the disease in each treated population to a statistically significant extent.

(d) Nerve and Spinal Cord Injuries

Populations of human patients with a confirmed clinical diagnosis of each of nerve and spinal cord injury will be administered a dose of 0.01-3.0 μg/kg of BMP-7 once weekly via a centrally located catheter in accordance with the methods of the present invention. It is expected that such treatment will modulate the disease in each treated population to a statistically significant.

(e) Tumor Metastasis

A population of human patients with a confirmed clinical diagnosis of tumor metastasis will be administered a dose of 0.01-3.0 μg/kg of BMP-7 once weekly via a centrally located catheter in accordance with the methods of the present invention. It is expected that such treatment will modulate the disease to a statistically significant extent in the treated patient population.

### EQUIVALENTS

The invention may be embodied in other specific forms without departing from the spirit or essential characteristics thereof. The present embodiments are therefore to be considered illustrative and not restrictive, the scope of the invention being indicated by the appended claims rather than by the foregoing description, and all changes which come within the meaning and range of equivalency of the claims are therefore intended to be embraced therein.

1. A method of treating a disease or an injured or diseased tissue in a patient by systemically administering a bone morphogenetic protein to a patient in need thereof, the method comprising the step of:
administering the bone morphogenetic protein to the patient at an administration site via a vascular access structure, wherein the bone morphogenetic protein is delivered to the patient at a centrally located delivery site in the patient.

2. The method of claim 1, further comprising the step of implanting a vascular access structure with central venous access in the patient.

3. The method of claim 2, wherein the central venous access is via the jugular vein, the subclavian vein, the superior vena cava, or the femoral vein.

4. The method of claim 1, wherein the vascular access structure is a central venous catheter or central venous port.

5. The method of claim 1, wherein the administration site is peripheral.

6. The method of claim 5, wherein the vascular access structure is a PICC line.

7. The method of claim 1, wherein the administration site is centrally located.

8. The method of claim 1, wherein the bone morphogenetic protein is BMP-7.

9. The method of claim 1, wherein the delivery site is substantially edema free and substantially non-perturbed.

10. The method of claim 1, wherein the vascular access structure is substantially healed in place prior to administration of the bone morphogenetic protein.

11.-21. (canceled)

22. The method of claim 1, wherein non-vascular tissue at, near or adjacent the delivery site is substantially free of biologic agent following delivery.

23. The method of claim 1, wherein the injured or diseased tissue is a non-mineralized tissue.

24. The method of claim 1, wherein the injured or diseased tissue is an organ.

25. The method of claim 1, wherein said biologic agent is bioavailable for at least about 2 hours.

26. The method of claim 1, wherein said effective amount is about 100 to about 300 micrograms of biologic agent.

27.-48. (canceled)

49. A method of treatment of an injured or diseased tissue, the method comprising the step of:
administering to an administration site a composition comprising a biologic agent, and
delivering to an intravascular delivery site the composition such that intima tissue integrity at the delivery site is substantially uncompromised
whereupon the biologic agent disperses from the delivery site at a rate and in an amount effective to treat the injured or diseased tissue.

50. The method of claim 49, wherein the administration site and the delivery site are the same.

51. The method of claim 49, wherein the delivery site is venular-valve-free.

52. The method of claim 49, wherein the dispersal rate is about 1 ml/min.

53. The method of claim 49, whereupon the delivering step is accomplished using an intravascular apparatus having a distal end with a non-damaging configuration.

54.-60. (canceled)
